(19) United States
(12) Patent Application Publication
(10) Pub. No.: US 2025/0069687 A1
(43) Pub. Date: Feb. 27, 2025

(54) CHARACTERIZATION OF WHOLE GENOME DUPLICATION IN A GENOMIC COHORT OF OVER 14000 CELL FREE DNA SAMPLES

(71) Applicant: GUARDANT HEALTH, INC., Palo Alto, CA (US)

(72) Inventors: Andrew M. GROSS, Cincinnati, OH (US); Hao WANG, Redwood City, CA (US); Denis TOLKUNOV, Dublin, CA (US); Colby JENKINS, Palo Alto, CA (US); Sara WIENKE, Charleston, SC (US); Catalin BARBACIORU, Fremont, CA (US); Han-Yu CHUANG, Redwood City, CA (US)

(21) Appl. No.: 18/439,682

(22) Filed: Feb. 12, 2024

Related U.S. Application Data
(60) Provisional application No. 63/484,702, filed on Feb. 13, 2023.

(51) Int. Cl. G16B 20/10; G16B 40/20

(52) U.S. Cl. CPC G16B 20/10; G16B 40/20

(57) ABSTRACT

Described herein are methods and compositions related to whole gene duplication (WGD). Methods are described for detecting and determining the presence of WGD in a sample, including cell free nucleic acids derived from a subject, such as a liquid sample (e.g., blood, plasma), as well as chromosomal instability and genomic alterations. In various embodiments, the aforementioned methods are used in diagnosis, prognosis and treatment. In other embodiments, processing of samples characterized by WGD is described to confer increased accuracy and precision of detection.

### CROSS REFERENCE TO OTHER APPLICATIONS

This application claims the benefit of U.S. Provisional Patent Application No. 63/484,702 filed Jul. 1, 2018, which is incorporated by reference herein in its entirety.

### FIELD OF THE INVENTION

Described herein are methods and compositions related to oncology, including detection, diagnosis, prognosis and treatment for events related to whole gene duplication (WGD).

### BACKGROUND

Wide-scale rates of whole genome duplication (WGD) have previously been demonstrated in studies performed in tissue among genomically characterized tumors. Such studies report varied rates of WGD depending on tumor type and stage. Hallmarks of WGD include widespread loss-of-heterozygosity as well as associations with tumor proliferation and specific oncogenic driver events such as TP53 mutations. It has been proposed that presence of WGD could be a prognostic biomarker, leading to interest in profiling this genomic landmark in large retrospective cohorts to inform future clinical studies. Bielski et al., “Genome doubling shapes the evolution and prognosis of advanced cancers” Nature Genetics volume 50:1189-1195 (2018). Lopez et al., “Interplay between whole-genome doubling and the accumulation of deleterious alterations in cancer evolution” Nature Genetics volume 52:3-293 (2020). For example, ploidy aware models drastically reduce false positive homdels and properly predict tumor fraction. However, there is a great need in the art to carry out WGD analysis in blood and cell-free nucleic acids, which has been hereto unachieved.

### SUMMARY OF THE INVENTION

Described herein is a method, including: obtaining nucleic acid sequence information from a sample derived from a subject, applying a log likelihood variant caller to the nucleic acid sequence information to generate a score, determining the presence of one or more genomes in the sample based on the score. In other embodiments, the log likelihood-based copy-number variant caller includes parameters for tumor purity and/or tumor ploidy. In other embodiments, determining the presence of one or more genomes includes applying normalized coverage and/or germline variant allele frequencies. In other embodiments, applying normalized coverage and/or germline variant allele frequencies is genome wide. In other embodiments, the score includes a ploidy count. In other embodiments, the sample includes cell-free DNA (cfDNA) In other embodiments, the one or more genomes is the result of whole genome doubling (WGD). In other embodiments, the methods includes administration of a therapeutic agent to the subject. In other embodiments, the therapeutic agent is selected on the basis of the determination of one or more genomes. In other embodiments, the log likelihood-based copy-number variant caller includes the formula in FIG. 1. In other embodiments, the nucleic acid sequence information includes sequencing a plurality of polynucleotides derived from the samples to generate a set of sequence reads, wherein the set of sequencing reads includes sequences of one or more molecular barcodes. In other embodiments, the one or more genomes is the result of whole genome doubling. In other embodiments, the method includes attaching nucleic acid adapters to nucleic acid sequences from the sample. In other embodiments, the nucleic acid adapters comprise molecular barcodes and/or are configured to generate molecular barcodes when attached to the nucleic acid sequences, and wherein the molecular barcodes identify a particular polynucleotide and/or single original cell-free nucleic acid molecules from the nucleic acid sequences using at least the sequences of the molecular barcodes, each including a polynucleotide that combine with the diversity of the sequence of the plurality of polynucleotides to identify the particular polynucleotide and/or single original cell-free nucleic acid molecule.

Described herein is a method including obtaining nucleic acid sequence information from a sample derived from a subject, determining the presence of one or more genomes in the sample. In other embodiments, the presence of one or more genomes in the sample includes measuring a signature. In other embodiments, the signature includes one or more of: CC1, UBR4, DNM1L, COPS4, PLEKHO2, CBR4, NUP43, FAM129B/NIBAN2, PSMD13, DUSP10, FAM13A, TRMT10A, ANAPC4, SGO1, TMEM170A, TUBG1, COPS2, SERPINE2, PCGF6, AP1S3, EXOSC3, MUC17, LRRC46, HSPH1, BIRC6, LARP7, SNRNP70, DHX8, INTS9, ENG, FERMT2, SPEN, EGFR, JAK1, MET, PRKCA, PI3KCA. BUB1B, ANLN, ARPC2, NCKAP1, VPS29, CELA2BM EIPR1M BCAR3, FUBP1, HGS, SPDYA, WDR26, SLC9A3R1, FLX3, SBDS, HECTD1, MICU1, NUP98, REXO2, and ARHGAP23. In other embodiments, the one or more genomes is the result of whole genome doubling (WGD). In other embodiments, the method includes administration of a therapeutic agent to the subject. In other embodiments, the method includes selecting the therapeutic agent based on a signature and/or whole genome doubling (WGD). In other embodiments, the presence of one or more genomes application of a log likelihood-based copy-number variant caller. In other embodiments, the log likelihood-based copy-numver variant caller includes the formula in FIG. 1. In other embodiments, the method includes attaching nucleic acid adapters to nucleic acid sequences from the sample. In other embodiments, the nucleic acid adapters comprise molecular barcodes and/or are configured to generate molecular barcodes when attached to the nucleic acid sequences, and wherein the molecular barcodes identify a particular polynucleotide and/or single original cell-free nucleic acid molecules from the nucleic acid sequences using at least the sequences of the molecular barcodes, each including a polynucleotide that combine with the diversity of the sequence of the plurality of polynucleotides to identify the particular polynucleotide and/or single original cell-free nucleic acid molecule.

Described herein is composition made by a process, including obtaining nucleic acid sequences from a sample derived from a subject, wherein the sample includes cell-free DNA (cfDNA), and attaching nucleic acid adapters to the nucleic acid sequences to generate the adapter attached nucleic acid sequences. In other embodiments, the composition includes adapter attached nucleic acid sequences includes one or more genomes. In other embodiments, the one or more genomes is the result of whole genome doubling (WGD). In other embodiments, the nucleic acid adapters comprise molecular barcodes and/or are configured to generate molecular barcodes when attached to the nucleic acid sequences, and wherein the molecular barcodes identify a particular polynucleotide and/or single original cell-free nucleic acid molecules from the nucleic acid sequences using at least the sequences of the molecular barcodes, each including a polynucleotide that combine with the diversity of the sequence of the plurality of polynucleotides to identify the particular polynucleotide and/or single original cell-free nucleic acid molecule.

### BRIEF DESCRIPTION OF FIGURES

FIG. 1. Likelihood-based copy-number variant caller. This model jointly selects tumor purity and ploidy while fitting normalized coverage and germline variant allele frequencies across genome-wide data.

FIG. 2. Baseline setting. As shown, a baseline can be set at similar location, but current version is centered properly.

FIG. 3. Ploidy aware model. As shown, tumor fraction prediction in ploidy-aware model is harmonious with coverage data.

FIG. 4. Error reduction. An exemplary reduction in false positive calls is illustrated.

FIG. 5. Ploidy measurement. To interpret polyploidism, tumor ploidy is 4, normal ploidy is 2, with a mixed baseline level that is normal+((tumor ploidy−tumor ploidy)*tumor_fraction). Further, clear evidence of polyploidy (balanced duplications at CN=4, CN=6, cct, 12%); mean copy number >=2.5 (32%); median copy number >=3 (24%); fraction of genome duplicated >50% (26%).

FIG. 6. TP53 mutated samples. As shown here, the relative maximum allele frequency (MAF) of TP53 compared to the tumor fraction is higher as shown.

### DETAILED DESCRIPTION

Despite interest in whole genome duplication (WGD), a central challenge in studying WGD in solid tumors has been identifying WGD events from what may be multiple successive and independent copy number amplifications (CNAs). Adding to complexity is WGD apparent role in subsequent chromosomal aberrations and genomic instability. Another challenge has been delineating the mutational correlates of WGD in a cohort of diverse cancer types of sufficient population size to draw robust inferences. Finally, due to the limited clinical outcomes data available for large-scale genomic cohorts, the clinical significance of WGD remains largely unexplored. WGD is therefore a common but still cryptic event in human cancers, the evolution and clinical impact of which has not yet been broadly defined in both common and rare cancers.

In cancer, whole genome duplication (WGD) significantly elevates genome instability. Such genome instability originates from DNA damage resulting from the incomplete mitosis that initiates the process of tetraploidization, a type of ploidy abnormality, replication stress, and DNA damage associated with an enlarged genome, and chromosomal instability during the subsequent mitosis in the presence of extra centrosomes and altered spindle morphology. Increased genome instability contributes to tumor progression in various ways, including (1) Increased Genetic Diversity: diversity within the cancer cell population contributes to tumor heterogeneity, which can increase the survival of subpopulations of cells with distinct genetic alterations, potentially making the tumor more adaptable to changing environments and treatments (2) Enhanced Tumor Cell Survival: Enhanced survival capability via increased resistance to apoptosis, can contribute to the persistence and expansion of tumor cells, even under environmental challenges such as therapeutic interventions. (3) Increased Chromosomal Instability: Chromosomal instability can drive the accumulation of additional genetic alterations (at a higher mutation rate), promoting the acquisition of traits associated with malignancy, such as increased invasiveness and metastatic potential. (4) Aneuploidy: Polyploid cells resulting from WGD may undergo subsequent events, such as chromosomal missegregation during cell division. This numerical imbalance in the chromosome count can lead to the generation of aneuploid daughter cells within the tumor, contributing to further genomic diversity and promoting tumor progression. (5) Altered Gene Expression Patterns: WGD can influence gene dosage and expression patterns, leading to altered cellular functions and providing a selective advantage for certain tumor cell populations, including aggressive tumor behavior. (6) Tumor Adaptation to Stressful Conditions: Polyploid cells may exhibit increased adaptability to challenging environmental conditions, such as nutrient deprivation or hypoxia. Adaptability to such conditions can enhance the survival and growth of tumor cells within the challenging microenvironments commonly found in tumors. (7) Tumor Evolution: WGD facilitates accelerated genomic evolution within the tumor cell population. This accelerated evolution can drive the selection of advantageous genetic variants, promoting the emergence of more aggressive and therapy-resistant tumor phenotypes over time.

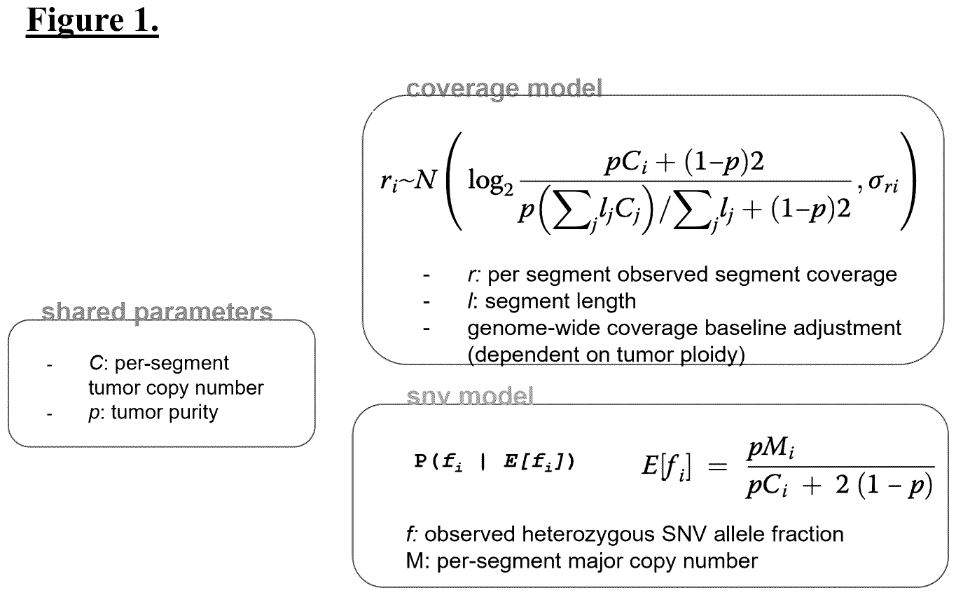

Described herein is a method, including: obtaining nucleic acid sequence information from a sample derived from a subject, applying a log likelihood variant caller to the nucleic acid sequence information to generate a score, determining the presence of one or more genomes in the sample based on the score. In other embodiments, the log likelihood-based copy-number variant caller includes parameters for tumor purity and/or tumor ploidy. In other embodiments, determining the presence of one or more genomes includes applying normalized coverage and/or germline variant allele frequencies. In other embodiments, applying normalized coverage and/or germline variant allele frequencies is genome wide. In other embodiments, the score includes a ploidy count. In other embodiments, the sample includes cell-free DNA (cfDNA) In other embodiments, the one or more genomes is the result of whole genome doubling (WGD). In other embodiments, the methods includes administration of a therapeutic agent to the subject. In other embodiments, the therapeutic agent is selected on the basis of the determination of one or more genomes. In other embodiments, the log likelihood-based copy-number variant caller includes the formula in FIG. 1. In other embodiments, the nucleic acid sequence information includes sequencing a plurality polynucleotides derived from the samples to generate a set of sequence reads, wherein the set of sequencing reads includes sequences of one or more molecular barcodes. In other embodiments, the one or more genomes is the result of whole genome doubling. In other embodiments, the method includes attaching nucleic acid adapters to nucleic acid sequences from the sample. In other embodiments, the nucleic acid adapters comprise molecular barcodes and/or are configured to generate molecular barcodes when attached to the nucleic acid sequences, and wherein the molecular barcodes identify a particular polynucleotide and/or single original cell-free nucleic acid molecules from the nucleic acid sequences using at least the sequences of the molecular barcodes, each including a polynucleotide that combine with the diversity of the sequence of the plurality of polynucleotides to identify the particular polynucleotide and/or single original cell-free nucleic acid molecule.

Described herein is a method including obtaining nucleic acid sequence information from a sample derived from a subject, determining the presence of one or more genomes in the sample. In other embodiments, the presence of one or more genomes in the sample includes measuring a signature. In other embodiments, the signature includes one or more of: CC1, UBR4, DNM1L, COPS4, PLEKHO2, CBR4, NUP43, FAM129B/NIBAN2, PSMD13, DUSP10, FAM13A, TRMT10A, ANAPC4, SGO1, TMEM170A, TUBG1, COPS2, SERPINE2, PCGF6, AP1S3, EXOSC3, MUC17, LRRC46, HSPH1, BIRC6, LARP7, SNRNP70, DHX8, INTS9, ENG, FERMT2, SPEN, EGFR, JAK1, MET, PRKCA, PI3KCA. BUB1B, ANLN, ARPC2, NCKAP1, VPS29, CELA2BM EIPR1M BCAR3, FUBP1, HGS, SPDYA, WDR26, SLC9A3R1, FLX3, SBDS, HECTD1, MICU1, NUP98, REXO2, and ARHGAP23. In other embodiments, the one or more genomes is the result of whole genome doubling (WGD). In other embodiments, the method includes administration of a therapeutic agent to the subject. In other embodiments, the method includes selecting the therapeutic agent based on a signature and/or whole genome doubling (WGD). In other embodiments, the presence of one or more genomes application of a log likelihood-based copy-number variant caller. In other embodiments, the log likelihood-based copy-numver variant caller includes the formula in FIG. 1. In other embodiments, the method includes attaching nucleic acid adapters to nucleic acid sequences from the sample. In other embodiments, the nucleic acid adapters comprise molecular barcodes and/or are configured to generate molecular barcodes when attached to the nucleic acid sequences, and wherein the molecular barcodes identify a particular polynucleotide and/or single original cell-free nucleic acid molecules from the nucleic acid sequences using at least the sequences of the molecular barcodes, each including a polynucleotide that combine with the diversity of the sequence of the plurality of polynucleotides to identify the particular polynucleotide and/or single original cell-free nucleic acid molecule. In various embodiments, the method includes obtaining sequencing information, including sequence reads, or sequencing nucleic acids derived from deoxyribonucleic acid (DNA) molecules of a cell-free bodily fluid sample of a subject; generating a first data set of the sequence information to generate one or more quantitative measures related to ploidy count of the sequencing information. In other embodiments, the first data set is transformed into a second data set by calculating the probability that the quantitative measure, results from whole genome doubling (WGD) and/or results from subsequent chromosomal aberrations and genomic instability. In other embodiments, the method includes determining for one or more sequenced nucleic acids, that each sequence is from any one of WGD, subsequent chromosomal aberration and genomic instability.

Described herein is composition made by a process, including obtaining nucleic acid sequences from a sample derived from a subject, wherein the sample includes cell-free DNA (cfDNA), and attaching nucleic acid adapters to the nucleic acid sequences to generate the adapter attached nucleic acid sequences. In other embodiments, the composition includes adapter attached nucleic acid sequences includes one or more genomes. In other embodiments, the one or more genomes is the result of whole genome doubling (WGD). In other embodiments, the nucleic acid adapters comprise molecular barcodes and/or are configured to generate molecular barcodes when attached to the nucleic acid sequences, and wherein the molecular barcodes identify a particular polynucleotide and/or single original cell-free nucleic acid molecules from the nucleic acid sequences using at least the sequences of the molecular barcodes, each including a polynucleotide that combine with the diversity of the sequence of the plurality of polynucleotides to identify the particular polynucleotide and/or single original cell-free nucleic acid molecule.

Obtaining Sequencing Reads from DNA Molecules of a Cell-Free Bodily Fluid from a Subject.

Obtaining sequencing reads from DNA molecules of a cell-free bodily fluid of a subject can comprise obtaining a cell-free bodily fluid. Exemplary cell-free bodily fluids are or can be derived from serum, plasma, blood, saliva, urine, synovial fluid, whole blood, lymphatic fluid, ascites fluid, interstitial or extracellular fluid, the fluid in spaces between cells, including gingival crevicular fluid, bone marrow, cerebrospinal fluid, saliva, mucous, sputum, semen, sweat, urine, or any other bodily fluids. A cell-free bodily fluid can be selected from the group consisting of plasma, urine, or cerebrospinal fluid. A cell-free bodily fluid can be plasma. A cell-free bodily fluid can be urine. A cell-free bodily fluid can be cerebrospinal fluid.

Nucleic acid molecules, including DNA molecules, can be extracted from cell-free bodily fluids. DNA molecules can be genomic DNA. DNA molecules can be from cells of healthy tissue of the subject. DNA molecules can be from noncancerous cells that have undergone somatic mutation. DNA molecules can be from a fetus in a maternal sample. The skilled worker will understand that, in embodiments wherein the DNA molecules are from a fetus in a maternal sample, a subject may refer to the fetus even though the sample is maternal. DNA molecules can be from precancerous cells of the subject. DNA molecules can be from cancerous cells of the subject. DNA molecules can be from cells within primary tumors of the subject. DNA molecules can be from secondary tumors of the subject. DNA molecules can be circulating DNA. The circulating DNA can comprise circulating tumor DNA (ctDNA). DNA molecules can be double-stranded or single-stranded. Alternatively, DNA molecule can comprise a combination of a double-stranded portion and a single-stranded portion. DNA molecules do not have to be cell-free. In some cases, the DNA molecules can be isolated from a sample. For example, DNA molecules can be cell-free DNA isolated from a bodily fluid, e.g., serum or plasma.

A sample can comprise various amounts of genome equivalents of nucleic acid molecules. For example, a sample of about 30 ng DNA can contain about 10,000 haploid human genome equivalents and, in the case of cfDNA, about 200 billion individual polynucleotide molecules. Similarly, a sample of about 100 ng of DNA can contain about 30,000 haploid human genome equivalents and, in the case of cfDNA, about 600 billion individual molecules.

Cell-free DNA molecules may be isolated and extracted from bodily fluids using a variety of techniques known in the art. In some cases, cell-free nucleic acids may be isolated, extracted and prepared using commercially available kits such as the Qiagen Qiamp® Circulating Nucleic Acid Kit protocol. In other examples, Qiagen Qubit™ dsDNA HS Assay kit protocol, Agilent™ DNA 1000 kit, or TruSeq™ Sequencing Library Preparation; Low-Throughput (LT) protocol may be used to quantify nucleic acids. Cell-free nucleic acids may be fetal in origin (via fluid taken from a pregnant subject), or may be derived from tissue of the subject itself. Cell-free nucleic acids can be derived from a neoplasm (e.g. a tumor or an adenoma).

Generally, cell-free nucleic acids are extracted and isolated from bodily fluids through a partitioning step in which cell-free nucleic acids, as found in solution, are separated from cells and other non-soluble components of the bodily fluid. Partitioning may include, but is not limited to, techniques such as centrifugation or filtration. In other cases, cells are not partitioned from cell-free nucleic acids first, but rather lysed. In one example, the genomic DNA of intact cells is partitioned through selective precipitation. Cell-free nucleic acids, including DNA, may remain soluble and may be separated from insoluble genomic DNA and extracted. Generally, after addition of buffers and other wash steps specific to different kits, nucleic acids may be precipitated using isopropanol precipitation. Further clean up steps may be used such as silica based columns to remove contaminants or salts. General steps may be optimized for specific applications. Non-specific bulk carrier nucleic acids, for example, may be added throughout the reaction to optimize certain aspects of the procedure such as yield.

Cell-free DNA molecules can be at most 500 nucleotides in length, at most 400 nucleotides in length, at most 300 nucleotides in length, at most 250 nucleotides in length, at most 225 nucleotides in length, at most 200 nucleotides in length, at most 190 nucleotides in length, at most 180 nucleotides in length, at most 170 nucleotides in length, at most 160 nucleotides in length, at most 150 nucleotides in length, at most 140 nucleotides in length, at most 130 nucleotides in length, at most 120 nucleotides in length, at most 110 nucleotides in length, or at most 100 nucleotides in length.

Cell-free DNA molecules can be at least 500 nucleotides in length, at least 400 nucleotides in length, at least 300 nucleotides in length, at least 250 nucleotides in length, at least 225 nucleotides in length, at least 200 nucleotides in length, at least 190 nucleotides in length, at least 180 nucleotides in length, at least 170 nucleotides in length, at least 160 nucleotides in length, at least 150 nucleotides in length, at least 140 nucleotides in length, at least 130 nucleotides in length, at least 120 nucleotides in length, at least 110 nucleotides in length, or at least 100 nucleotides in length. In particular, cell-free nucleic acids can be between 140 and 180 nucleotides in length.

Cell-free DNA can comprise DNA molecules from healthy tissue and tumors in various amounts. Tumor-derived cell-free DNA can be at least 0.1% of the total amount of cell-free DNA in the sample, at least 0.2% of the total amount of cell-free DNA in the sample, at least 0.5% of the total amount of cell-free DNA in the sample, at least 0.7% of the total amount of cell-free DNA in the sample, at least 1% of the total amount of cell-free DNA in the sample, at least 2% of the total amount of cell-free DNA in the sample, at least 3% of the total amount of cell-free DNA in the sample, at least 4% of the total amount of cell-free DNA in the sample, at least 5% of the total amount of cell-free DNA in the sample, at least 10% of the total amount of cell-free DNA in the sample, at least 15% of the total amount of cell-free DNA in the sample, at least 20% of the total amount of cell-free DNA in the sample, at least 25% of the total amount of cell-free DNA in the sample, or at least 30% of the total amount of cell-free DNA in the sample, or more.

In some cases, DNA molecules can be sheared during the extraction process and comprise fragments between 100 and 400 nucleotides in length. In some cases, nucleic acids can be sheared after extraction can comprise nucleotides between 100 and 400 nucleotides in length. In some cases, DNA molecules are already between 100 and 400 nucleotides in length and additional shearing is not purposefully implemented.

A subject can be an animal. A subject can be a mammal, such as a dog, horse, cat, mouse, rat, or human. A subject can be a human. A subject can be suspected of having cancer. A subject can have previously received a cancer diagnosis. The cancer status of a subject may be unknown. A subject can be male or female. A subject can be at least 20 years old, at least 30 years old, at least 40 years old, at least 50 years old, at least 60 years old, or at least 70 years old.

Sequencing may be by any method known in the art. For example, sequencing techniques include classic techniques (e.g., dideoxy sequencing reactions (Sanger method) using labeled terminators or primers and gel separation in slab or capillary) and next generation techniques. Exemplary techniques include sequencing by synthesis using reversibly terminated labeled nucleotides, pyrosequencing, 454 sequencing, Illumina/Solexa sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, SOLID sequencing targeted sequencing, single molecule real-time sequencing, exon sequencing, electron microscopy-based sequencing, panel sequencing, transistor-mediated sequencing, direct sequencing, random shotgun sequencing, whole-genome sequencing, sequencing by hybridization, capillary electrophoresis, gel electrophoresis, duplex sequencing, cycle sequencing, single-base extension sequencing, solid-phase sequencing, high-throughput sequencing, massively parallel signature sequencing, emulsion PCR, co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, real-time sequencing, reverse-terminator sequencing, nanopore sequencing, MS-PET sequencing, and a combination thereof. In some embodiments, the sequencing method is massively parallel sequencing, that is, simultaneously (or in rapid succession) sequencing any of at least 100, 1000, 10,000, 100,000, 1 million, 10 million, 100 million, or 1 billion polynucleotide molecules. In some embodiments, sequencing can be performed by a gene analyzer such as, for example, gene analyzers commercially available from Illumina or Applied Biosystems. Sequencing of separated molecules has more recently been demonstrated by sequential or single extension reactions using polymerases or ligases as well as by single or sequential differential hybridizations with libraries of probes. Sequencing may be performed by a DNA sequencer (e.g., a machine designed to perform sequencing reactions). In some embodiments, a DNA sequencer can comprise or be connected to a database, for example, that contains DNA sequence data.

A sequencing technique that can be used includes, for example, use of sequencing-by-synthesis systems. In the first step, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to DNA capture beads, e.g., streptavidin-coated beads using, e.g., Adaptor B, which contains 5′-biotin tag. The fragments attached to the beads are PCR amplified within droplets of an oil-water emulsion. The result is multiple copies of clonally amplified DNA fragments on each bead. In the second step, the beads are captured in wells (pico-liter sized). Pyrosequencing is performed on each DNA fragment in parallel. Addition of one or more nucleotides generates a light signal that is recorded by a CCD camera in a sequencing instrument. The signal strength is proportional to the number of nucleotides incorporated. Pyrosequencing makes use of pyrophosphate (PPi) which is released upon nucleotide addition. PPi is converted to ATP by ATP sulfurylase in the presence of adenosine 5′ phosphosulfate. Luciferase uses ATP to convert luciferin to oxyluciferin, and this reaction generates light that is detected and analyzed.

Another example of a DNA sequencing technique that can be used is SOLID technology by Applied Biosystems from Life Technologies Corporation (Carlsbad, Calif.). In SOLID sequencing, genomic DNA is sheared into fragments, and adaptors are attached to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations are prepared in microreactors containing beads, primers, template, and PCR components. Following PCR, the templates are denatured and beads are enriched to separate the beads with extended templates. Templates on the selected beads are subjected to a 3′ modification that permits bonding to a glass slide. The sequence can be determined by sequential hybridization and ligation of partially random oligonucleotides with a central determined base (or pair of bases) that is identified by a specific fluorophore. After a color is recorded, the ligated oligonucleotide is removed and the process is then repeated.

Another example of a DNA sequencing technique that can be used is ion semiconductor sequencing using, for example, a system sold under the trademark ION TORRENT by Ion Torrent by Life Technologies (South San Francisco, Calif.). Ion semiconductor sequencing is described, for example, in Rothberg, et al., An integrated semiconductor device enabling non-optical genome sequencing, Nature 475:348-352 (2011); U.S. Pub. 2010/0304982; U.S. Pub. 2010/0301398; U.S. Pub. 2010/0300895; U.S. Pub. 2010/0300559; and U.S. Pub. 2009/0026082, the contents of each of which are incorporated by reference in their entirety.

Another example of a sequencing technology that can be used is Illumina sequencing. Illumina sequencing is based on the amplification of DNA on a solid surface using fold-back PCR and anchored primers. Genomic DNA is fragmented, and adapters are added to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection and identification steps are repeated. Sequencing according to this technology is described in U.S. Pat. Nos. 7,960,120; 7,835,871; 7,232,656; 7,598,035; 6,911,345; 6,833,246; 6,828,100; 6,306,597; 6,210,891; U.S. Pub. 2011/0009278; U.S. Pub. 2007/0114362; U.S. Pub. 2006/0292611; and U.S. Pub. 2006/0024681, each of which are incorporated by reference in their entirety.

Another example of a sequencing technology that can be used includes the single molecule, real-time (SMRT) technology of Pacific Biosciences (Menlo Park, Calif.). In SMRT, each of the four DNA bases is attached to one of four different fluorescent dyes. These dyes are phospholinked. A single DNA polymerase is immobilized with a single molecule of template single stranded DNA at the bottom of a zero-mode waveguide (ZMW). It takes several milliseconds to incorporate a nucleotide into a growing strand. During this time, the fluorescent label is excited and produces a fluorescent signal, and the fluorescent tag is cleaved off. Detection of the corresponding fluorescence of the dye indicates which base was incorporated. The process is repeated.

Another example of a sequencing technique that can be used is nanopore sequencing (Soni & Meller, 2007, Progress toward ultrafast DNA sequence using solid-state nanopores, Clin Chem 53 (11): 1996-2001). A nanopore is a small hole, of the order of 1 nanometer in diameter. Immersion of a nanopore in a conducting fluid and application of a potential across it results in a slight electrical current due to conduction of ions through the nanopore. The amount of current which flows is sensitive to the size of the nanopore. As a DNA molecule passes through a nanopore, each nucleotide on the DNA molecule obstructs the nanopore to a different degree. Thus, the change in the current passing through the nanopore as the DNA molecule passes through the nanopore represents a reading of the DNA sequence.

Another example of a sequencing technique that can be used involves using a chemical-sensitive field effect transistor (chemFET) array to sequence DNA (for example, as described in U.S. Pub. 2009/0026082). In one example of the technique, DNA molecules can be placed into reaction chambers, and the template molecules can be hybridized to a sequencing primer bound to a polymerase. Incorporation of one or more triphosphates into a new nucleic acid strand at the 3′ end of the sequencing primer can be detected by a change in current by a chemFET. An array can have multiple chemFET sensors. In another example, single nucleic acids can be attached to beads, and the nucleic acids can be amplified on the bead, and the individual beads can be transferred to individual reaction chambers on a chemFET array, with each chamber having a chemFET sensor, and the nucleic acids can be sequenced.

Another example of a sequencing technique that can be used involves using an electron microscope as described, for example, by Moudrianakis, E. N. and Beer M., in Base sequence determination in nucleic acids with the electron microscope, III. Chemistry and microscopy of guanine-labeled DNA, PNAS 53:564-71 (1965). In one example of the technique, individual DNA molecules are labeled using metallic labels that are distinguishable using an electron microscope. These molecules are then stretched on a flat surface and imaged using an electron microscope to measure sequences.

Prior to sequencing, adaptor sequences can be attached to the nucleic acid molecules and the nucleic acids can be enriched for particular sequences of interest. Sequence enrichment can occur before or after the attachment of adaptor sequence.

The nucleic acid molecules or enriched nucleic acid molecules can be attached to any sequencing adaptor suitable for use on any sequencing platform disclosed herein. For example, a sequence adaptor can comprise a flow cell sequence, a sample barcode, or both. In another example, a sequence adaptor can be a hairpin shaped adaptor, a Y-shaped adaptor, a forked adaptor, and/or comprise a sample barcode. In some cases, the adaptor does not comprise a sequencing primer region. In some cases the adaptor-attached DNA molecules are amplified, and the amplification products are enriched for specific sequences as described herein. In some cases, the DNA molecules are enriched for specific sequences after preparing a sequencing library. Adaptors can comprise barcode sequence. The different barcode can be at least 1, 2, 3, 4, 5, 6, 7, 8, 9, 10, 11, 12, 13, 14, 15, 16, 17, 18, 19, 20, 21, 22, 23, 24, 25, or more (or any length as described throughout) nucleic acid bases, e.g., 7 bases. The barcodes can be random sequences, degenerate sequences, semi-degenerate sequences, or defined sequences. In some cases, there is a sufficient diversity of barcodes that substantively (e.g., at least 70%, at least 80%, at least 90%, or at least 99% of) each nucleic acid molecule is tagged with a different barcode sequence. In some cases, there is a sufficient diversity of barcodes that substantively (e.g., at least 70%, at least 80%, at least 90%, or at least 99% of) each nucleic acid molecule from a particular genetic locus is tagged with a different barcode sequence.

A sequencing adaptor can comprise a sequence capable of hybridizing to one or more sequencing primers. A sequencing adaptor can further comprise a sequence hybridizing to a solid support, e.g., a flow cell sequence. For example, a sequencing adaptor can be a flow cell adaptor. The sequencing adaptors can be attached to one or both ends of a polynucleotide fragment. In another example, a sequencing adaptor can be hairpin shaped. For example, the hairpin shaped adaptor can comprise a complementary double-stranded portion and a loop portion, where the double-stranded portion can be attached (e.g., ligated) to a double-stranded polynucleotide. Hairpin shaped sequencing adaptors can be attached to both ends of a polynucleotide fragment to generate a circular molecule, which can be sequenced multiple times.

In some cases, none of the library adaptors contains a sample identification motif (or sample molecular barcode). Such sample identification motif can be provided via sequencing adaptors. A sample identification motif can include a sequencer of at least 4, 5, 6, 7, 8, 9, 10, 20, 30, or 40 nucleotide bases that permits the identification of polynucleotide molecules from a given sample from polynucleotide molecules from other samples. For example, this can permit polynucleotide molecules from two subjects to be sequenced in the same pool and sequence reads for the subjects subsequently identified.

A sequencer motif includes nucleotide sequence(s) needed to couple a library adaptor to a sequencing system and sequence a target polynucleotide coupled to the library adaptor. The sequencer motif can include a sequence that is complementary to a flow cell sequence and a sequence (sequencing initiation sequence) that can be selectively hybridized to a primer (or priming sequence) for use in sequencing. For example, such sequencing initiation sequence can be complementary to a primer that is employed for use in sequence by synthesis (e.g., Illumina). Such primer can be included in a sequencing adaptor. A sequencing initiation sequence can be a primer hybridization site.

In some cases, none of the library adaptors contains a complete sequencer motif. The library adaptors can contain partial or no sequencer motifs. In some cases, the library adaptors include a sequencing initiation sequence. The library adaptors can include a sequencing initiation sequence but no flow cell sequence. The sequence initiation sequence can be complementary to a primer for sequencing. The primer can be a sequence specific primer or a universal primer. Such sequencing initiation sequences may be situated on single-stranded portions of the library adaptors. As an alternative, such sequencing initiation sequences may be priming sites (e.g., kinks or nicks) to permit a polymerase to couple to the library adaptors during sequencing.

Adaptors can be attached to DNA molecules by ligation. In some cases, the adaptors are ligated to duplex DNA molecules such that each adaptor differently tags complementary strands of the DNA molecule. In some cases, adaptor sequences can be attached by PCR, wherein a first portion of a single-stranded DNA is complementary to a target sequence and a second portion comprises the adaptor sequence.

Enrichment for particular sequences of interest can be performed by sequence capture methods. Sequence capture can be performed using immobilized probes that hybridize to the targets of interest. Sequence capture can be performed using probes attached to functional groups, e.g., biotin, that allow probes hybridized to specific sequences to be enriched for from a sample by pulldown. In some cases, prior to hybridization to functionalized probes, specific sequences such as adaptor sequences from library fragments can be masked by annealing complementary, non-functionalized polynucleotide sequences to the fragments in order to reduce non-specific or off-target binding. Sequence probes can target specific genes. Sequence capture probes can target specific genetic loci or genes. Such genes can be oncogenes.

Sequence capture probes can tile across a gene (e.g., probes can target overlapping regions). Sequence probes can target non-overlapping regions. Sequence probes can be optimized for length, melting temperature, and secondary structure.

### Quantitative Measures of Guanine-Cytosine (GC) Content

Guanine-cytosine content is the percentage of nitrogenous bases of a DNA molecule that are either guanine or cytosine. A quantitative measure related to GC content for a genetic locus can be the GC content of the entire genetic locus. A quantitative measure related to GC content for a genetic locus can be the GC content of the exonic regions of the gene. A quantitative measure related to GC content for a genetic locus can be the GC content of the regions covered by reads mapping to the genetic locus. A quantitative measure related to GC content can be the GC content of the sequence capture probes corresponding to the genetic locus. A quantitative measure related to GC content for a genetic locus can be a measure related to central tendency of the GC content of the sequence capture probes corresponding to the genetic locus. The measure related to central tendency can be any measure of central tendency such as mean, median, or mode. The measure related to central tendency can be the median. GC content of a given region can be measured by dividing the number of guanosine and cytosine bases by the total number of bases over that region.

### Quantitative Measures of Sequencing Read Coverage

A quantitative measure related to sequencing read coverage is a measure indicative of the number of reads derived from a DNA molecule corresponding to a genetic locus (e.g., a particular position, base, region, gene or chromosome from a reference genome). In order to associate reads to a genetic locus, the reads can be mapped or aligned to the reference. Software to perform mapping or aligning (e.g., Bowtie, BWA, mrsFAST, BLAST, BLAT) can associate a sequencing read with a genetic locus. During the mapping process, particular parameters can be optimized. Non-limiting examples of optimization of the mapping processing can include masking repetitive regions; employing mapping quality (e.g., MAPQ) score cut-offs; using different seed lengths to generate alignments; and limiting the edit distance between positions of the genome.

Quantitative measures associated with sequencing read coverage can include counts of reads associated with a genetic locus. In some cases, the counts are transformed into new metrics to mitigate the effects of differing sequencing depth, library complexity, or size of the genetic locus. Exemplary metrics are Read Per Kilobase per Million (RPKM), Fragments Per Kilobase per Million (FPKM), Trimmed Mean of M values (TMM), variance stabilized raw counts, and log transformed raw counts. Other transformations are also known to those of skill in the art that may be used for particular applications.

Quantitative measures can be determined using collapsed reads, wherein each collapsed read corresponds to an initial template DNA molecule. Methods to collapse and quantify read families are found in PCT/US2013/058061 and PCT/US2014/000048, each of which is herein incorporated by reference in its entirety. In particular, collapsing methods can be employed that use barcodes and sequence information from the sequencing read to collapse reads into families, such that each family shares barcode sequences and at least a portion of the sequencing read sequence. Each family is then, for the majority of the families, derived from a single initial template DNA molecule. Counts derived from mapping sequences from families can be referred to as “unique molecular counts” (UMCs). In some cases, determining a quantitative measure related to sequencing read coverage comprises normalizing UMCs by a metric related to library size to provide normalized UMCs (“normalized UMCs”). Exemplary methods are dividing the UMC of a genetic locus by the sum of all UMCs; dividing the UMC of a genetic locus by the sum of all autosomal UMCs. When comparing multiple sequencing read data sets, UMCs can, for example, be normalized by the median UMCs of the genetic loci of the two sequencing read data sets. In some cases, the quantitative measure related to sequencing read coverage can be normalized UMCs that are further normalized as follows: (i) normalized UMCs are determined for corresponding genetic loci from sequencing reads derived from training samples; (ii) for each genetic locus, normalized UMCs of the sample are normalized by the median of the normalized UMCs of the training samples at the corresponding loci, thereby providing Relative Abundances (RAs) of genetic loci.

Consensus sequences can identified based on their sequences, for example by collapsing sequencing reads based on identical sequences within the first 5, 10, 15, 20, or 25 bases. In some cases, collapsing allows for 1 difference, 2 differences, 3 differences, 4 differences, or 5 differences in the reads that are otherwise identical. In some cases, collapsing uses the mapping position of the read, for example the mapping position of the initial base of the sequencing read. In some cases, collapsing uses barcodes, and sequencing reads that share barcode sequences are collapsed into a consensus sequence. In some cases, collapsing uses both barcodes and the sequence of the initial template molecules. For example, all reads that share a barcode and map to the same position in the reference genome can be collapsed. In another example, all reads that share a barcode and a sequence of the initial template molecule (or a percentage identity to a sequence of the initial template molecule) can be collapsed.

In some cases, quantitative measures of sequencing read coverage are determined for specific sub-regions of a genome. Regions can be bins, genes of interest, exons, regions corresponding to sequence probes, regions corresponding to primer amplification products, or regions corresponding to primer binding sites. In some cases, sub-regions of the genome are regions corresponding to sequence capture probes. A read can map to a region corresponding to the sequence capture probe if at least a portion of the read maps at least a portion of the region corresponding to the sequence capture probe. A read can map to a region corresponding to the sequence capture probe if at least a portion of the read maps to the majority of the region corresponding to the sequence capture probe. A read can map to a region corresponding to the sequence capture probe if at least a portion of the read maps across the center point of the region corresponding to the sequence capture probe. In some cases, a quantitative measure related to sequencing read coverage of a genetic locus is the median of the RAs of the probes corresponding to genomic locations within the genetic locus. For example, if KRAS is covered by three probes, which have RAs of 2, 3, and 5, the RA of the genetic locus would be 3.

### Computers

The present disclosure provides computer control systems that are programmed to implement methods of the disclosure. Described herein is a computer system that is programmed or otherwise configured to implement methods of the present disclosure. The computer system includes a central processing unit (CPU, also “processor” and “computer processor” herein), which can be a single core or multi core processor, or a plurality of processors for parallel processing. The computer system also includes memory or memory location (e.g., random-access memory, read-only memory, flash memory), electronic storage unit (e.g., hard disk), communication interface (e.g., network adapter) for communicating with one or more other systems, and peripheral devices, such as cache, other memory, data storage and/or electronic display adapters. The memory, storage unit, interface and peripheral devices are in communication with the CPU through a communication bus (solid lines), such as a motherboard. The storage unit can be a data storage unit (or data repository) for storing data. The computer system can be operatively coupled to a computer network (“network”) with the aid of the communication interface. The network can be the Internet, an internet and/or extranet, or an intranet and/or extranet that is in communication with the Internet. The network in some cases is a telecommunication and/or data network. The network can include a local area network. The network can include one or more computer servers, which can enable distributed computing, as cloud computing. The network, in some cases with the aid of the computer system, can implement a peer-to-peer network, which may enable devices coupled to the computer system 1201 to behave as a client or a server.

The CPU can execute a sequence of machine-readable instructions, which can be embodied in a program or software. The instructions may be stored in a memory location, such as the memory. The instructions can be directed to the CPU, which can subsequently program or otherwise configure the CPU to implement methods of the present disclosure. Examples of operations performed by the CPU can include fetch, decode, execute, and writeback.

The CPU can be part of a circuit, such as an integrated circuit. One or more other components of the system can be included in the circuit. In some cases, the circuit is an application specific integrated circuit (ASIC).

The storage unit can store files, such as drivers, libraries and saved programs. The storage unit can store user data, e.g., user preferences and user programs. The computer system in some cases can include one or more additional data storage units that are external to the computer system, such as located on a remote server that is in communication with the computer system through an intranet or the Internet.

The computer system can communicate with one or more remote computer systems through the network. For instance, the computer system can communicate with a remote computer system of a user.

Methods as described herein can be implemented by way of machine (e.g., computer processor) executable code stored on an electronic storage location of the computer system, such as, for example, on the memory or electronic storage unit. The machine executable or machine readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system can include or be in communication with an electronic display that comprises a user interface (UI) for providing, for example, a report. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit.

### Methods of Treatment, Diagnosis and Prognosis

Described herein is a method of treating a subject with a cancer comprising one or more whole genome duplication, also known as whole genome doubling (WGD), events. In exemplary aspects, the method comprises: detecting whole genome doubling in the subject, administering a therapeutic agent to the subject In other embodiments, the method is a method of treating a subject with a cancer comprising one or more whole genome duplication or whole genome doubling (WGD) events characterized by a signature, and administering a therapeutic agent to the subject. In various embodiments, detecting whole genome doubling, includes obtaining sequencing information from the subject, applying a likelihood-based copy-number variant caller to the sequence information, measuring WGD to determine the therapeutic agent administered to the subject. In various embodiments, the likelihood-based copy-number variant caller includes tumor purity and/or tumor ploidy. In various embodiments, measuring WGD includes normalized coverage and germline variant allele frequencies. In various embodiments, measuring WGD including fitting normalized coverage and germline variant allele frequencies across genome-wide data.

Biomarkers involved in WGD signatures can include markers of associated with tolerance to whole genome duplication or PI3K/AKT-mediated tolerance of whole genome duplication. This may include, for example, one or more of: RCC1, UBR4, DNM1L, COPS4, PLEKHO2, CBR4, NUP43, FAM129B/NIBAN2, PSMD13, DUSP10, FAM13A, TRMT10A). The therapy may inhibit PARP1 and the signature may be a signature associated with impaired homologous recombination. This may include, for example, one or more of: ANAPC4, SGO1, TMEM170A, TUBG1, COPS2, SERPINE2, PCGF6, AP1S3, EXOSC3, MUC17, LRRC46, HSPH1, BIRC6, LARP7, SNRNP70, DHX8, INTS9, ENG, FERMT2, SPEN. The therapy may inhibit a kinase in a mitogenic pathway. This may include, for example, one or more of: EGFR, JAK1, MET, PRKCA, PI3KCA). This may include, for example, a signature associated with replication stress. This may include, for example, one or more of: BUB1B, ANLN, ARPC2, NCKAP1, VPS29, CELA2BM EIPR1M BCAR3, FUBP1, HGS, SPDYA, WDR26, SLC9A3R1, FLX3, SBDS). The therapy may inhibit CDK4 and the signature may be a signature associated with replication stress. This may include, for example, one or more of: HECTD1, MICU1, NUP98, REXO2, ARHGAP23.

The methods described herein may be used to provide a prognosis for a subject. Thus, based on the determination step, a subject may be classified as having a good or a poor prognosis, where prognosis is known to be associated with exposure to one or more signatures. For example, a signature may be known to be associated with prognosis if samples with a high or low exposure to the one or more signatures are associated with different prognosis in a cohort of patients. In other words, a signature may be known to be associated with prognosis if samples with a good prognosis in a cohort of patients have a significantly different expected exposure to the signature than samples with a poor prognosis. Whether a prognosis is considered good or poor may vary between cancers and stage of disease. In general terms a good prognosis is one where the overall survival (OS), disease free survival (DFS) and/or progression-free survival (PFS) is longer than that of a comparative group or value, such as e.g. the average for that stage and cancer type. A prognosis may be considered poor if OS, DFS and/or PFS is lower than that of a comparative group or value, such as e.g. the average for that stage and type of cancer.

### Example 1—Methods

Samples were selected that were sequentially run as part of lab clinical offerings on a high content genomic profiling assay using cell-free DNA extracted from blood. Only samples originating from individuals with primary breast, colorectal, or prostate cancer were analyzed due to limited sample size in other indications. The Inventors deployed a likelihood-based copy-number variant caller, which jointly selects tumor purity and ploidy while fitting normalized coverage and germline variant allele frequencies across genome-wide data as shown in FIG. 1. Related information can be found in Riester et al., “PureCN: copy number calling and SNV classification using targeted short read sequencing” Source Code for Biology and Medicine 11:13 (2016), which is incorporated by reference herein.

Briefly, this approach assesses likelihood of each segment given per sample purity, ploidy, baseline status and asserts that total copy number is constant between coverage and SNV model. Due to the limited resolution of the test, only samples where we predicted to have greater than 10% tumor fraction from copy number variant (CNV) profiles were considered for statistical analysis. The presence of whole genome duplication was assessed by counting the median major chromosomal copy number (maximum of allele specific copy numbers). Presence of SNV/InDels were annotated, assessed for pathogenicity, and filtered to those with a pathogenic allele frequency greater than 10%.

More specifically, one can center coverage for broadening out allele-frequency usage. The applied model is independent of tumor purity, wherein one models coverage as (1/MAF) and take peaks in distribution of offsets and test candidates in likelihood model. This approach assumes a majority of sample is reference or simple deletion/duplication (not CN-LOH), high-copy duplication and homozygous deletions are in the minority, and baseline is diploid (can be adjusted post-hoc for higher ploidy).

### Example 2—Results

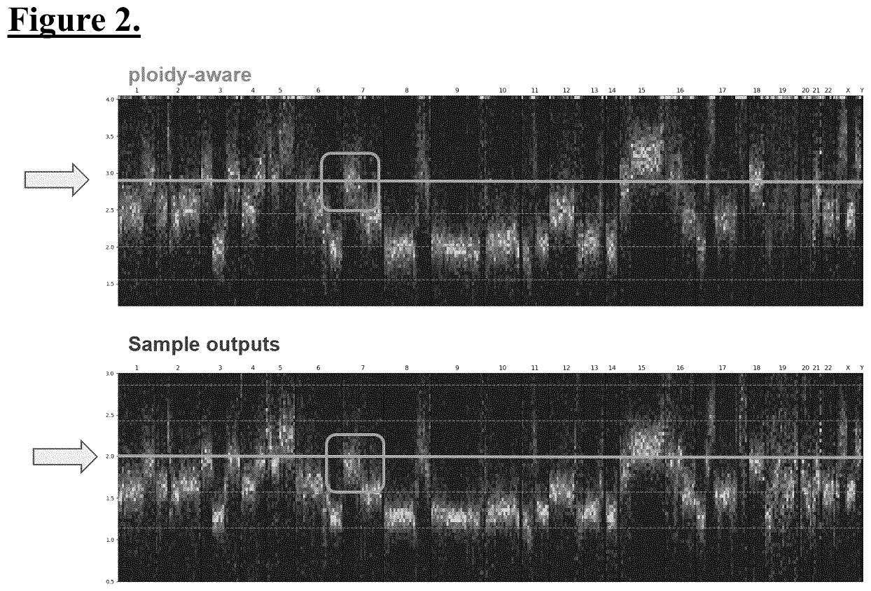

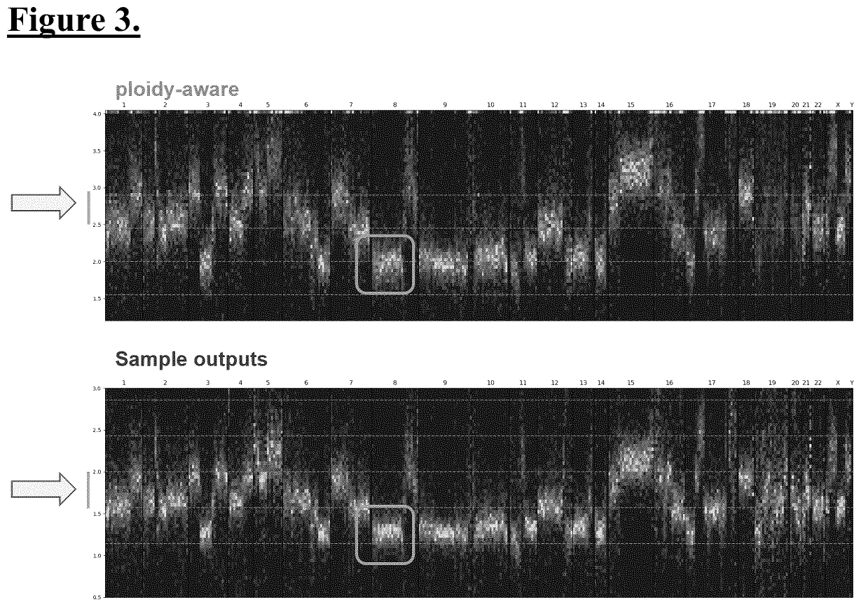

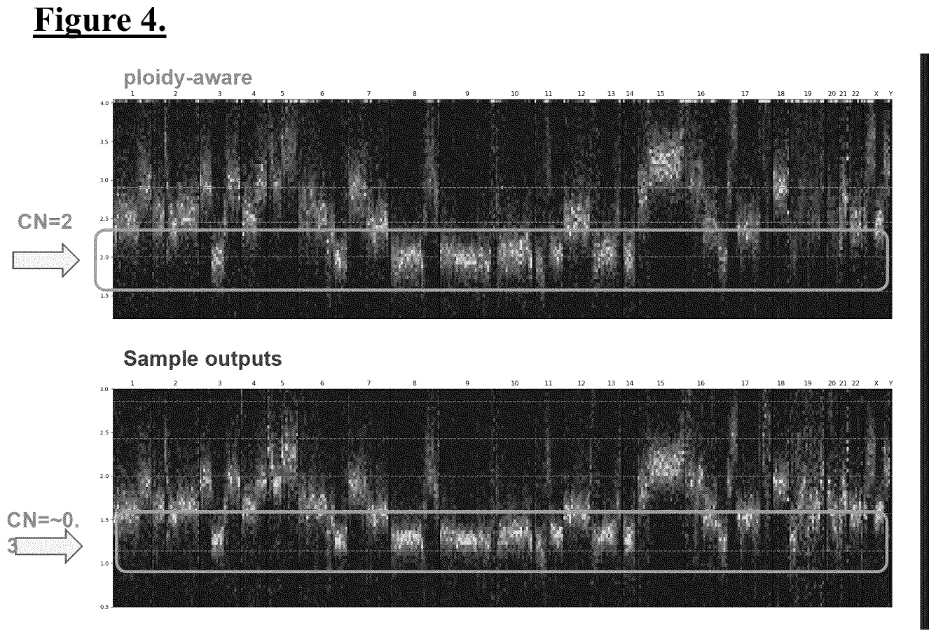

As an example of modeling, show is FIG. 2, wherein a baseline set at similar location, but current version is centered properly. In FIG. 3, tumor fraction prediction in ploidy-aware model is harmonious with coverage data. In. FIG. 4, an exemplary reduction in false positive calls is illustrated.

The aforementioned approach already applies overfitting by assuming all center states are diploid, given that tumor ploidy prediction is commonly reported with tumor purity. Decreased false positive homdel rate is large enough to offset any tradeoffs. This includes for example, the results in Table 1.

TABLE 1

| sample | 0 | 1-9 | 10+ |
|---|---|---|---|
| 0 | 346 | 27 | 5 |
| 1-9 | 72 | 129 | 3 |
| 10+ | 41 | 18 | 26 |

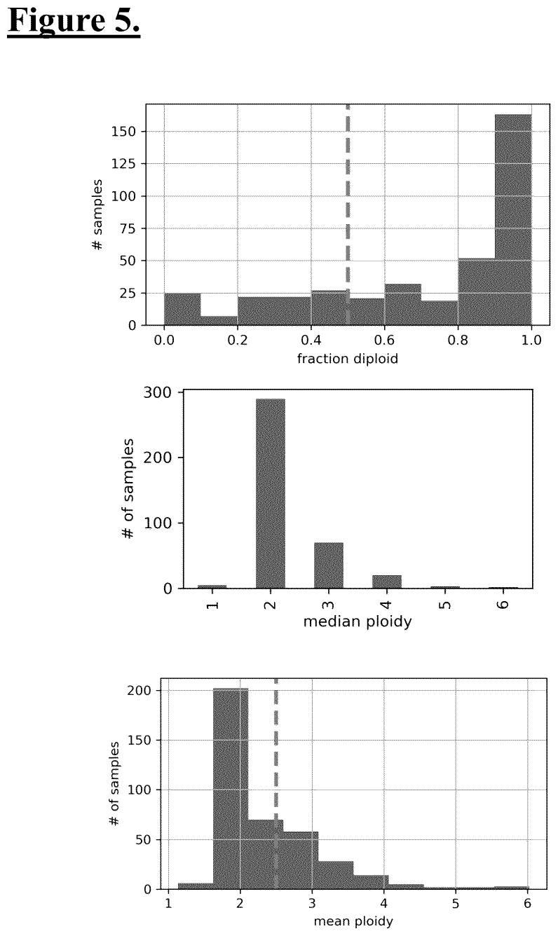

To interpret polyploid sample, it can be interpreted that tumor ploidy is 4, normal ploidy is 2, with a mixed baseline level that is normal+((tumor ploidy−tumor ploidy)*tumor_fraction). Further, clear evidence of polyploidy (balanced duplications at CN=4, CN=6, ect, 12%); mean copy number >=2.5 (32%); median copy number >=3 (24%); fraction of genome duplicated >50% (26%). An example of each is shown in FIG. 5.

### Example 4—Further Results

A total of 14,076 samples were analyzed, with 5362 passing the 10% observed tumor fraction cutoff. WGD was annotated in 2195 (41%) samples with varying rates across sub-cohorts (Table 1). Somatic mutation in TP53 was associated with a large increase in rate of WGD (odds ratio 6.9) which is consistent but higher in magnitude than previously reported associations. Amplification of CCNE1 and homozygous deletion of genes including WRN and RB1 were also associated with WGD, independent of TP53 mutation status and overall rates of aneuploidy.

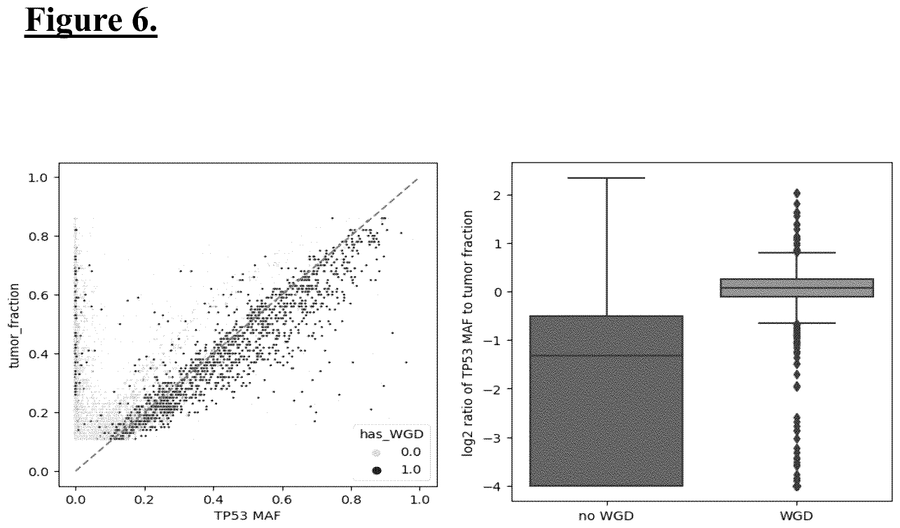

In addition to the overlap of WGD with TP53 mutations, there is also a clear trend with TP53 mutated samples that the relative MAF of TP53 compared to the tumor fraction is higher as shown in FIG. 6.

### Example 5—Sample Findings

WGD was detected in 41% of samples with greater than 10% tumor fraction in cell-free DNA across three cancer types shown in Table 2. While this represents a substantial percentage of advanced cancers, more research needs to be done regarding the impact of these events on clinical outcomes and treatment response. Additionally, co-occurrence of these events with other advanced cancer biomarkers such as microsatellite instability or homologous recombination deficiency was not reported. Given the high rates of these events, future retrospective studies are warranted to correlate presence of WGD with patient outcomes and response.

TABLE 2

| | All samples | | | | Samples with | |
|---|---|---|---|---|---|---|
| | | no | has | | TP53 mut | |
| tissue | n | WGD | WGD | frac_WGD | n | frac_WGD |
| Breast | 1821 | 1260 | 561 | 0.31 | 419 | 0.67 |
| Colorectal | 2009 | 919 | 1090 | 0.54 | 1215 | 0.71 |
| Prostate | 1532 | 988 | 544 | 0.36 | 429 | 0.61 |

All references cited herein are incorporated by reference. The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

1. A method, comprising:
obtaining nucleic acid sequence information from a sample derived from a subject;
applying a log likelihood variant caller to the nucleic acid sequence information to generate a score; and
determining the presence of one or more genomes in the sample based on the score.

2. The method of claim 1, wherein the log likelihood-based copy-number variant caller comprises parameters for tumor purity and/or tumor ploidy.

3. The method of claim 1, wherein determining the presence of one or more genomes comprises applying normalized coverage and/or germline variant allele frequencies.

4. The method of claim 3, wherein the applying normalized coverage and/or germline variant allele frequencies is genome wide.

5. The method of claim 1, wherein the score comprises a ploidy count.

6. The method of claim 1, wherein the sample comprises cell-free DNA (cfDNA).

7. The method of claim 1, wherein the one or more genomes is the result of whole genome doubling (WGD).

8. The method of claim 1, comprising administration of a therapeutic agent to the subject.

9. The method of claim 9, wherein the therapeutic agent is selected on the basis of the determination of one or more genomes.

10. The method of claim 1, wherein the log likelihood-based copy-number variant caller comprises the formula in FIG. 1.

11. The method of claim 1, wherein obtaining nucleic acid sequence information comprises sequencing a plurality polynucleotides derived from the samples to generate a set of sequence reads, wherein the set of sequencing reads comprises sequences of one or more molecular barcodes.

12. The method of claim 1, wherein the one or more genomes is the result of whole genome doubling.

13. A method, comprising:
obtaining nucleic acid sequence information from a sample derived from a subject; and
determining the presence of one or more genomes in the sample.

14. The method of claim 13, wherein determining the presence of one or more genomes in the sample comprises measuring a signature.

15. The method of claim 14, wherein the signature comprises one or more of: CC1, UBR4, DNM1L, COPS4, PLEKHO2, CBR4, NUP43, FAM129B/NIBAN2, PSMD13, DUSP10, FAM13A, TRMT10A, ANAPC4, SGO1, TMEM170A, TUBG1, COPS2, SERPINE2, PCGF6, AP1S3, EXOSC3, MUC17, LRRC46, HSPH1, BIRC6, LARP7, SNRNP70, DHX8, INTS9, ENG, FERMT2, SPEN, EGFR, JAK1, MET, PRKCA, PI3KCA, BUB1B, ANLN, ARPC2, NCKAP1, VPS29, CELA2BM EIPR1M BCAR3, FUBP1, HGS, SPDYA, WDR26, SLC9A3R1, FLX3, SBDS, HECTD1, MICU1, NUP98, REXO2, and ARHGAP23.

16. The method of claim 13, wherein the one or more genomes is the result of whole genome doubling (WGD).

17. The method of claim 13, comprising administration of a therapeutic agent to the subject.

18. The method of claim 17, comprising: selecting the therapeutic agent based on a signature and/or whole genome doubling (WGD).

19. The method of claim 13, wherein determining the presence of one or more genomes application of a log likelihood-based copy-number variant caller.

20. The method of claim 19, wherein the log likelihood-based copy-numver variant caller comprises the formula in FIG. 1.

21. A composition made by a process, comprising:
obtaining nucleic acid sequences from a sample derived from a subject, wherein the sample comprises cell-free DNA (cfDNA); and
attaching nucleic acid adapters to the nucleic acid sequences to generate the adapter attached nucleic acid sequences.

22. The composition of claim 21, wherein the composition comprises adapter attached nucleic acid sequences comprises one or more genomes.

23. The composition of claim 22, wherein the one or more genomes is the result of whole genome doubling (WGD).

24. The composition of claim 21, wherein the nucleic acid adapters comprise molecular barcodes and/or are configured to generate molecular barcodes when attached to the nucleic acid sequences, and wherein the molecular barcodes identify a particular polynucleotide and/or single original cell-free nucleic acid molecules from the nucleic acid sequences using at least the sequences of the molecular barcodes, each comprising a polynucleotide that combine with the diversity of the sequence of the plurality of polynucleotides to identify the particular polynucleotide and/or single original cell-free nucleic acid molecule.
